(19) United States
(12) Patent Application Publication
(10) Pub. No.: US 2026/0114891 A1
(43) Pub. Date: Apr. 30, 2026

(54) TISSUE SEPARATING DEVICE AND ASSOCIATED SYSTEMS AND METHODS

(71) Applicant: BOSTON SCIENTIFIC SCIMED, INC., Maple Grove, MN (US)

(72) Inventors: Alexander R. Cohen, Austin, TX (US); Ryan Davis Gilkey, Austin, TX (US)

(73) Assignee: BOSTON SCIENTIFIC SCIMED, INC., Maple Grove, MN (US)

(21) Appl. No.: 19/368,341

(22) Filed: Oct. 24, 2025

Related U.S. Application Data
(60) Provisional application No. 63/711,947, filed on Oct. 25, 2024.

(51) Int. Cl. A61B 17/32; A61B 17/34

(52) U.S. Cl. CPC A61B 17/32; A61B 17/3421; A61B 2017/320044; A61B 2017/3456

(57) ABSTRACT

Devices, systems, and methods for separating tissue by moving a material layer with respect to a tissue-separating end effector. The material layer is moved over a contact surface of the tissue-separating end effector. Contact of the material and the contact surface with tissue, with the moving material moving with respect to the contacted tissue, causes separation of contacted tissue. The tissue-separating end effector may be considered a blunt tissue dissector.

### CROSS-REFERENCE TO RELATED APPLICATIONS

This application claims the benefit of priority under 35 U.S.C. § 119 to U.S. Provisional Application No. 63/711,947, filed October 25, 2024, the entire disclosure of which is hereby incorporated by reference herein for all purposes.

### FIELD

The present disclosure relates generally to the field of medical devices, systems, and methods for separating tissue. More particularly, the present disclosure relates to medical devices, systems, and methods using a blunt device to separate or dissect tissue.

### BACKGROUND

Various medical procedures involve cutting, dissecting, resecting, excising, etc., anatomical structures such as biological tissue. Some medical procedures involve differentiating tissue layers with different anatomical, structural, etc., properties. For instance, procedures such as endoscopic submucosal dissection (ESD), endoscopic mucosal resection (EMR), and Peroral Endoscopic Myotomy (POEM), involve removing a lesion from one layer of tissue of an anatomical structure in the gastrointestinal (GI) system having more than one type of tissue layer. Typically, a fluid (e.g., saline) is injected at the treatment site to elevate / lift one layer of tissue with respect to another layer of tissue (to create a “bleb”) to facilitate cutting of a lesion in one of the tissue layers, such as with a knife. For instance, a lifting agent may be injected into the submucosal layer to separate the mucosal layer from the muscularis layer. A cutting knife may then be used to cut through the submucosa. However, the use of a sharp cutting instrument to separate the mucosa and submucosa may risk inadvertent cutting of tissue which is not intended to be cut, or inadvertent cutting of blood vessels which may cause undesired bleeding. Moreover, the submucosal tissue layer is formed of connective tissue which may, in some instances, be separated by a blunt instrument rather than a sharp cutting instrument. Various blunt dissection tools exist, but require multiple components or steps for use. Typically, current blunt dissection tools act less locally, and therefore tend to be difficult to control in smaller spaces. Also, blunt dissection tools typically do not have an element with an active means of separation, but, instead, simply are pushed through material using a blunted tip. Accordingly, there remains a need for improved devices, methods, and systems for cutting or dissecting or tunneling through tissue and/or separating tissue from other tissue, such as for separating one layer of tissue away from an adjacent layer of tissue, and/or for advancing a medical instrument through a tissue without the use of a sharp cutting instrument. Solutions to these and other challenges in the art would be welcome, and itis with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a tissue separating device is disclosed as having a tissue-separating end effector extending along a longitudinal axis. In some aspects, the tissue separator device includes an outer tubular elongate member having a distal end along the tissue-separating end effector of the tissue separating device and a proximal end; an inner tubular elongate member axially translatable within the outer tubular elongate member and having a distal end and a proximal end; a layer of material having a periphery coupled to the outer tubular elongate member and a radially-inward portion radially inward of the periphery and extending over the distal end of the inner tubular elongate member; and a tissue-separating tip element defining a contact surface extending transverse to the longitudinal axis. In some aspects, movement of the inner tubular elongate member with respect to the outer tubular elongate member moves the layer of material with respect to the outer tubular elongate member and the tissue-separating tip element; and movement of the layer of material with respect to the contact surface of the tissue-separating tip element causes separation of tissue contacted by the layer of material along the contact surface.

In some aspects, the periphery of the layer of material is fixedly coupled with respect to the outer tubular elongate member.

In some aspects, the device further includes a control handle operably associated with the proximal end of the outer tubular elongate member and the proximal end of the inner tubular member. In some aspects, the control handle includes an actuator operably coupled to the inner tubular elongate member to axially translate the inner tubular member with respect to the outer tubular elongate member to cause the layer of material to move with respect to the inner tubular elongate member.

In some aspects, the device further includes an elongate return element coupled to the radially-inward portion of the layer of material and configured to pull the layer of material proximally as the inner tubular elongate member is moved proximally within the outer tubular elongate member. In some aspects, the elongate return element defines a lumen therethrough and extending through the layer of material.

In some aspects, the tissue-separating end effector further includes a tissue-separating tip element. In some aspects, the tissue-separating tip element defines a blunt end defining the contact surface of the tissue-separating end effector. In some aspects, the tissue-separating tip element is provided along a distalmost end of the outer tubular elongate member. In some aspects, the tissue-separating tip element is separate from the distalmost end of the outer tubular elongate member.

In accordance with various principles of the present disclosure, a tissue separating device with a tissue-separating end effector extending along a longitudinal axis includes an outer tubular elongate member having a distal end along the tissue-separating end effector of the tissue separating device and a proximal end; an inner tubular elongate member axially translatable within the outer tubular elongate member and having a distal end and a proximal end; a layer of material extending over the distal ends of the outer tubular elongate member and the inner tubular elongate member; a tissue-separating tip element defining a contact surface extending transverse to the longitudinal axis; and an elongate return element having a distal end coupled to the layer of material and a proximal end. In some aspects, distal movement of the inner tubular member moves the layer of material distally and with respect to the tissue-separating tip element; the inner tubular member is movable proximally without moving the layer of material proximally; and proximal movement of the elongate return element moves the layer of material distally and with respect to the contact surface of the tissue-separating tip element to cause separation of tissue contacted by the layer of material along the contact surface.

In some aspects, the layer of material is fixedly attached with respect to the inner tubular member and movable with respect to the outer tubular member.

In some aspects, the device further comprising a control handle operably associated with the proximal end of the outer tubular elongate member, the proximal end of the inner tubular member, and the proximal end of the elongate return element. In some aspects, the control handle further includes a first actuator block operably associated with the proximal end of the inner tubular member, and a second actuator block operably associated with the proximal end of the elongate return element and movable with respect to the first actuator block. In some aspects, movement of the layer of material with respect to the contact surface of the tissue-separating tip element and movement of the second actuator block is faster than movement of the first actuator block.

In accordance with various principles of the present disclosure, a tissue separator includes a first portion of a layer of material fixedly coupled with respect to an actuator movable to move the layer of material with respect to a second portion of the layer of material.

In accordance with various principles of the present disclosure, a method of separating tissue includes providing a tissue separator having a tissue-separating end effector with a tissue-separating tip element and a layer of material having a first portion fixedly coupled to a first element of the tissue-separator and a second portion fixedly coupled to a second element of the tissue-separator; and moving the first element relative to the second element to move the layer of material with respect to the tissue-separating tip element.

In some aspects, the method further includes axially moving the second element with respect to the first element to move the layer of material with respect to the tissue-separating tip element. In some aspects, the first element is an outer tubular elongate member, the tissue separator further having an inner tubular elongate member movable with respect to the outer tubular elongate member, the method further including moving the inner tubular elongate member distally with respect to the outer tubular elongate member to move the layer of material from within a lumen of the inner tubular elongate member to outside the inner tubular elongate member, and moving the second element proximally with respect to the outer tubular elongate member to retract the layer of material into the inner tubular elongate member. In some aspects, the method further includes moving the layer of material distally from a proximal end of the tissue separator to a distal end of the tissue separator, over a tissue contact surface of the tissue-separating tip element, and proximally to the proximal end of the tissue separator. In some aspects, the first portion of the layer of material is fixedly coupled to the first element and the second portion of the layer of material is fixedly coupled to the second element, the method further comprising moving the first element proximally with respect to the second element to move the layer of material over and with respect to the tissue-separating tip element.

In various embodiments described or otherwise within the scope of the present disclosure, a tissue-separating end effector may have other configurations. In accordance with various principles of the present disclosure, a tissue separating device has a tissue-separating end effector defining a tissue contact surface with a first dimension greater than a second dimension. In some aspects, the tissue contact surface extends along an axis transverse to the longitudinal axis of the shaft. An elongated layer of material is movable over the elongated contact surface to define a blunt tissue separating element of the device. The elongated contact surface of the tissue-separating end effector allows selective orientation of tissue separation along a selected axis or plane. In some aspects, an elongated layer of material is moved continuously in a first direction with respect to tissue to effect tissue separation, and/or reciprocating distally and proximally with respect to the tissue to effect tissue separation. In some aspects, the elongated layer of material extends from a first end to a second end, with the elongated layer of material coiled about at least one of the first end or the second end. In some aspects, the elongated layer of material is wound into coil or wound off of a coil as the strip or band of material advances with respect to tissue to separate the tissue. In some aspects, the elongated layer of material forms a continuous loop.

In accordance with various principles of the present disclosure, a tissue separating device with a tissue-separating end effector extending along a longitudinal axis includes an elongate member, an elongated material layer (e.g. strip, band, belt, etc.) extending along the elongate member, and a tissue-separating tip element at the distal end of the elongate member, defining a contact surface transverse to the longitudinal axis.

In some aspects, the elongated material layer is movable with respect to the elongate member and the tissue-separating tip element.

In some aspects, the elongate member defines a lumen therethrough. In some aspects, the elongated material layer extends through lumen defined through the elongate member. In some aspects, the elongated material layer extends from within the lumen, out of the lumen, over the contact surface of the tissue-separating tip element, and back into the lumen.

In some aspects, the elongated material layer has a width equal to or less than the diameter of the elongate member. In some aspects, the elongated material layer extends from a first end to a second end. In some aspects, the first end and the second end of the elongated material layer are coupled to different actuator components. In some aspects, the first end and the second end of the elongated material layer are wound around spools/axles.

In some aspects, the device includes a proximal control handle. In some aspects, an end of the elongated material layer extends into the proximal control handle. In some aspects, movement of the elongated material layer is controlled by an actuator at the control handle.

In some aspects, the tissue-separating tip element is formed by crimping the distal end of the elongate member. In some aspects, the crimped distal end of the elongate member creates a flatter contact surface. In some aspects, the elongate member is tubular and defines a lumen therethrough, and cutouts are formed proximal to the crimped distal end for movement of the elongated material layer therethrough and into the lumen.

In some aspects, two elongated material layers move across the contact surface in opposite directions, causing tissue separation by elongating and breaking tissue fibers.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a device and system formed in accordance with various aspects of the present disclosure for separating tissue.

FIG. 2 illustrates a cross-sectional view of the tissue separator device illustrated in FIG. 1, and in a first configuration.

FIG. 3 illustrates a cross-sectional view of the tissue separator device illustrated in FIG. 2, but in a second configuration.

FIGS. 4A and 4B illustrate use of a system such as illustrated in FIG. 1.

FIG. 5 illustrates a tissue separator device such as illustrated in FIG. 2 and FIG. 3, but in a third configuration.

FIG. 6 illustrates a perspective view of another example of an embodiment of a device and system formed in accordance with various principles of the present disclosure.

FIG. 7 illustrates a perspective view of the tissue separator device illustrated in FIG. 6.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Devices, systems, and methods described herein for separating anatomical / biological tissue (e.g., within a tissue layer, and/or from an adjacent tissue layer) use a blunt end effector with a layer of material movable over the distal end of the blunt end effector to selectively move tissue, such as to dissect an area of tissue. It will be appreciated that terms such as dissect, resect, cut, excise, incise, tunnel into, snap, separate, etc., including other grammatical forms thereof, are usable interchangeably herein without intent to limit, unless otherwise specified. The tissue may be readily separable without a sharp cutting instrument. The devices, systems, and methods described herein may be configured for use in performing a procedure using open surgery (accessing the interior of a patient’s body by cutting open the body) or minimally invasive surgery (e.g., percutaneously, laparoscopically, endoscopically, etc.). For instance, devices, systems, and methods disclosed herein may be configured for use in procedures such as third space endoscopy (also known as submucosal endoscopy), which may access deeper layers of tissue within the body (e.g., the gastrointestinal (GI) tract) by tunneling into the tissue, such as between structurally differentiated layers of tissue. In the GI system, tunneling may be performed in the submucosal space with an endoscope, without compromising the integrity of the overlying mucosa. In some aspects, devices, systems, and methods of the present disclosure are used to dissect connective tissue of the submucosal space in a GI tract. In some aspects, devices, systems, and methods may be used to selectively dissect tissue, such as to selectively dissect only submucosal tissue. For instance, devices, systems, and methods of the present disclosure may be used for endoluminal submucosal dissection (ESD) procedures, Peroral Endoscopic Myotomy (POEM) procedures, and other procedures in which blunt dissection may be effective. Mechanically separating tissue using a blunt end effector in accordance with various principles of the present disclosure, such as described herein, provides various benefits, such as reducing potential for bleeding, improving pace of dissection, reducing risk of delayed bleeding, reducing risks associated with electrocautery, reducing risks of unintended damage which may be caused by a sharp element, and other benefits.

In accordance with various principles of the present disclosure, devices, systems, and methods of the present disclosure may be used to create long (e.g., over the full extent of a treatment area), controlled submucosal tunnels with relative ease, without risks associated with currently available devices and systems (e.g., inadvertent cutting beyond a target area). In some aspects, multiple tunnels can be made in series and/or in parallel to dissect large lesions without the use of a knife (e.g., blade, laser, electrocautery knife, etc.), to cut the tissue to clear a target area of tissue. In some aspects, a knife may be used to clear the vasculature that remains and was not cut by a tissue separator as disclosed herein. In some aspects, a coagulation device, such as an electrocautery device used on or in conjunction with a knife, may be included with (e.g., delivered with) the system to quickly manage vessels remaining after use of the devices, systems, and methods disclosed herein, including managing any inadvertent bleeding caused by cutting blood vessels. It will be appreciated that even if vasculature or other tissue or anatomical structures remain to be cut by another device than a tissue separator as disclosed herein, such structures are more visible than before use of a tissue separator as disclosed herein, having been aggregated during the dissection of the submucosal layer performed by the tissue separator.

In accordance with various principles of the present disclosure, movement of a sheet or layer of material is used to effect tissue separation. It will be appreciated that reference to terms such as a “sheet” or “layer” of material is intended to convey dimensions along two axes (e.g., perpendicular axes, such as x and y axes) which are greater than dimensions along a third axis (e.g., perpendicular to the first two axes, such as a z axis) when the material is generally planar, without limiting the material to extending in a single plane during use or otherwise. For instance, a material formed into the shape of a tube may still be considered a “sheet” or “layer” of material. In some aspects, the material is a film, a membrane, a fabric, etc, which may be formed from polyethylene terephthalate (PET), nylon, polytetrafluoroethylene (PTFE), polyethylene (PE), etc.. In some aspects, the film may be formed of a (preferably biocompatible) polymer or copolymer, such as polyethylene terephthalate (“PET”). In some aspects, the material has a coefficient of friction which causes sufficient abrasion of tissue to dissect the tissue. In some aspects, the movement of the sheet of material with respect to (e.g., over, in contact with, etc.) tissue mechanically tensions the fibers of the tissue, breaking such fibers to thus separate or cut the tissue. Devices and systems of the present disclosure have been found not to cut through certain types of tissue, such as mucosal or muscularis tissue layers of the GI tract, and thus do not inadvertently cut other types of tissue, such as blood vessels. Additionally or alternatively, devices and systems of the present disclosure have been found to facilitate a desired amount of displacement of tissue to separate of tissues, such as submucosal tissue from mucosal or muscularis tissue, without inadvertently tearing or damaging tissue. For instance, mechanical tensioning of the fibers of submucosal tissue snaps or breaks the fibers to separate tissue on one side of the tissue separator from tissue on the other side of the tissue separator. In some aspects, the sheet of material is moved with respect to tissue in a pulling apart fashion to stretch the tissue like rubber bands until the tissue snaps or breaks. In some aspects, larger vessels may be pushed out of the way of the sheet of material of the tissue separator without being snapped or cut. In some aspects, the tissue separation is achieved in a continuous motion (e.g., in the same distal or proximal direction). Additionally or alternatively, the sheet of material can be reciprocated (advanced and retracted, such as pulsed). In some aspects, the reciprocation is performed quickly, such as to act like a submucosal hedge trimmer. In some aspects, the sheet of material is deployable over longer controlled lengths.

Various embodiments of tissue moving devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element).

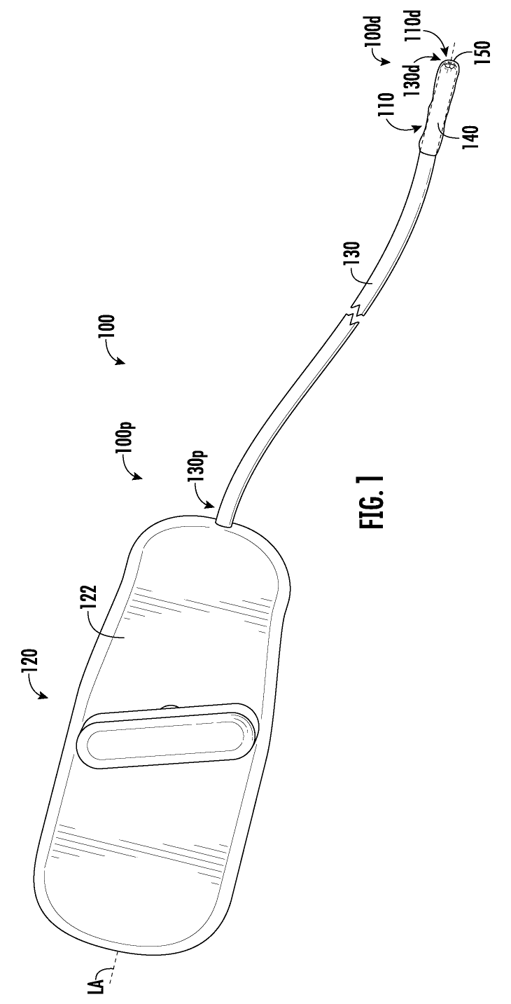

Turning now to the drawings, an example of an embodiment of a tissue-separating system 100 formed in accordance with various principles of the present disclosure is illustrated in FIG. 1. The tissue separating system 100 has a distal tissue-separating end 100d with a tissue-separating end effector 110, a proximal control end 100p with a control handle 120, and a tubular elongate member 130 (e.g., a sheath, catheter, etc.) extending between the distal tissue-separating end 100d and the proximal control end 100p. In some aspects, the tubular elongate member 130 (and other elements extending therein) is sufficiently flexible to be navigated within and through tortuous passages within a patient’s body, such as to perform a transluminal, transcatheter, endoluminal, etc., procedure with the tissue separating system 100. In some aspects, the distal end 130d of the tubular elongate member 130 forms a portion of the distal tissue-separating end 100d of the tissue separating system 100. More particularly, in some aspects, the distal end 130d of the tubular elongate member 130 is an element of the tissue-separating end effector 110 of the tissue separating system 100.

In accordance with various principles of the present disclosure, the tissue-separating end effector 110 utilizes a sheet of material, referenced herein as a material layer 140, which moves (e.g., with respect to the tubular elongate member 130) to cause tissue contacted or otherwise engaged by the material layer 140 to be separated. More particularly, the tissue-separating end effector 110 includes a tissue-separating tip element 150 defining a contact surface 152 over which the material layer 140 is moved. The material layer 140 (with the underlying contact surface 152) is advanced into contact with tissue, and the material layer 140 is moved with respect to the tissue to separate the tissue. In some aspects, the material layer 140 is moved with respect to the contact surface 152 as well as with respect to the tissue to separate the tissue. In some aspects, the contact surface 152 of the tissue-separating tip element 150 extends in a direction transverse to the longitudinal axis LA of the tissue separating system 100 and the tissue-separating end effector 110. For instance, in some aspects, the contact surface 152 of the tissue-separating tip element 150 extends in a plane transverse to the longitudinal axis LA of the tissue-separating system 100. As used herein, “transverse” is intended to include acute and obtuse angles other than 0° or 180°, unless otherwise specified. In some aspects, the material layer 140 may be considered to separate tissue extending along (e.g., substantially parallel to) the contact surface 152 of the tissue-separating tip element 150.

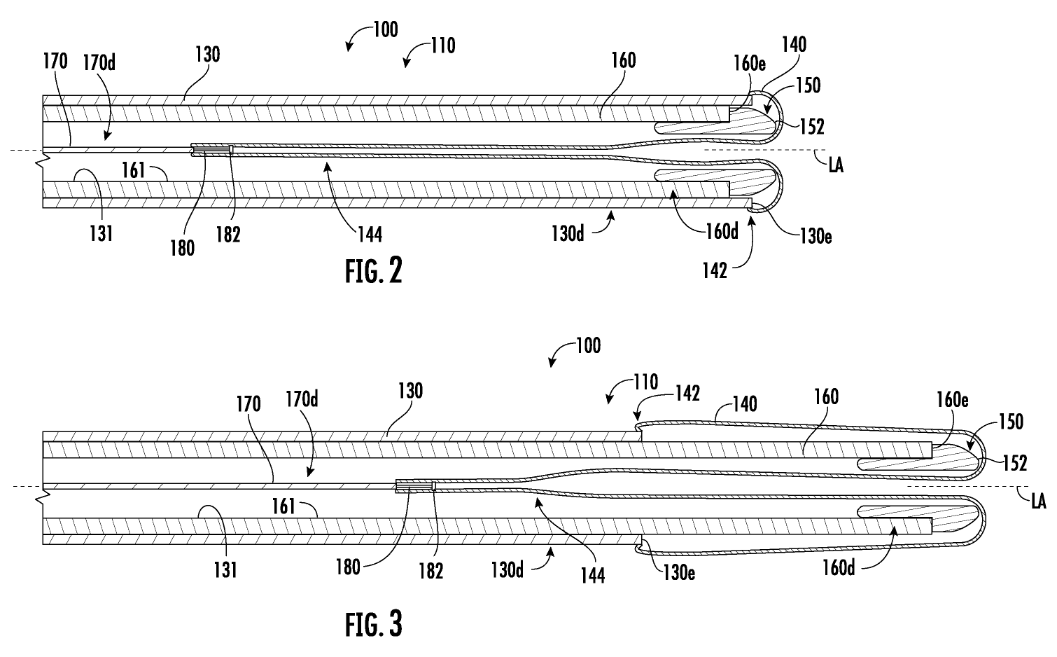

In some aspects, the tissue-separating tip element 150 is defined by the distalmost end 160e of a tubular elongate member 160 extending through the tubular elongate member 130. It will be appreciated that the tubular elongate member 130 may alternatively be referenced herein as an outer tubular elongate member 130 and the tubular elongate member 160 extending through the outer tubular elongate member 130 may be referenced herein as an inner tubular elongate member 160 to more clearly differentiate these elements from each other. In some aspects, a separate element with a blunt and/or rounded distalmost end (such as defining the contact surface 152 of the tissue-separating tip element 150) is provided as the tissue-separating tip element 150, such as illustrated in FIG. 2. Contact of the material layer 140 extending along the tissue-separating tip element 150 (such as, in particular, along the contact surface 152) with tissue as the material layer 140 moves with respect to the contact surface 152 of the tissue-separating tip element 150 causes the material layer 140 to separate tissue on one side of the tissue-separating tip element 150 from tissue on the other side of the tissue-separating tip element 150.

In some aspects, the example of an embodiment of a tissue-separating end effector 110 illustrated in FIG. 1 may be considered to include several elements, including the distal end 130d of the outer tubular elongate member 130, the material layer 140, and the distal end 160d of the inner tubular elongate member 160. In some aspects, the contact surface 152 of the tissue-separating tip element 150, positioned to contact tissue to be separated, has at least one dimension smaller than a corresponding dimension of (e.g., extending substantially coextensively with, though not the same extent as) the outer tubular elongate member 130. For instance, in some aspects, the outer tubular elongate member 130 and the tissue-separating tip element 150 have generally circular cross-sections, the outer diameter of the tissue-separating tip element 150 having a diameter smaller than the outer diameter of the outer tubular elongate member 130. In some aspects, the contact surface 152 is sized, shaped, configured, and/or dimensioned to present an element which is considered by those of ordinary skill in the art to be blunt, at least relative to knife or other cutting edge, and which alone would not cut tissue, but which provides a sufficiently small surface area to separate tissue upon movement of the material layer 140 with respect thereto. For instance, the contact surface 152 may be convexly curved, such as to provide a surface tangent to another surface. The smaller dimension of the tissue-separating tip element 150 and the contact surface 152 may facilitate tissue separation by the material layer 140 as the material layer 140 moves with respect to the contact surface 152 and contacts tissue to be separated. In some aspects, movement of the material layer 140 over the contact surface 152 creates a local advantage over tissue being encountered under tension by the tissue-separating system 100, such as by virtue of the smaller surface area of the contact surface 152 relative to other components of the tissue-separating system 100, and/or the movement and change of direction as the material layer 140 extends from within the interior of the tissue-separating tip element 150, across the contact surface 152, and over the exterior of the tissue-separating tip element 150.

In the example of an embodiment of a tissue separating system 100 illustrated in FIG. 1, the inner tubular elongate member 160 is axially translatable within the tubular elongate member 130 along a longitudinal axis LA (e.g., of the tissue separating system 100, the tissue-separating tip element 150, and/or along which the inner tubular elongate member 160 and/or the outer tubular elongate member 130 extends). The material layer 140 may be operatively associated with respect to the inner tubular elongate member 160 and the outer tubular elongate member 130 to be moved as the inner tubular elongate member 160 and the outer tubular elongate member 130 move with respect to each other. More particularly, in the illustrated example of an embodiment, the inner tubular elongate member 160 is translated (advanced or retracted) within and with respect to the outer tubular elongate member 130 to cause the material layer 140 to move with respect to the tissue-separating tip element 150 along the moving distal end 160d of the inner tubular elongate member 160. In some aspects, the movement of the material layer 140 over the tissue-separating tip element 150 mechanically tensions tissue contacted by the tissue-separating tip element 150 (such as, specifically, by the contact surface 152) to separate or tear the tissue. For instance, the movement of the material layer 140 over the tissue-separating tip element 150 may achieve a rolling action of the material layer 140 (e.g., like a conveyor belt or tractor wheel) which, when contacted with tissue such as submucosal tissue, causes the fibers of the of tissue to snap or break.

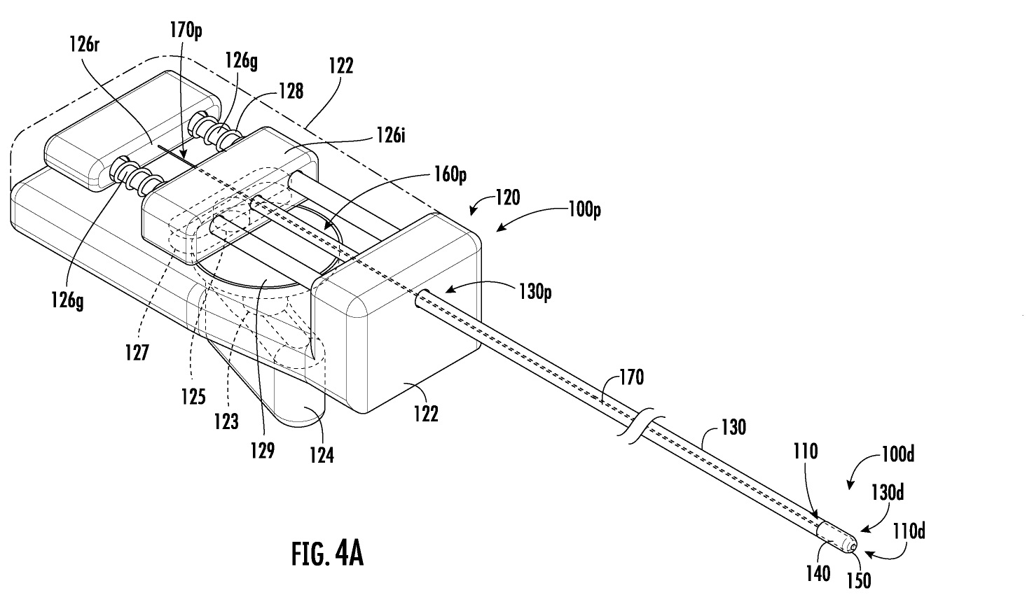

In some aspects, the material layer 140 may be considered to move from a stowed configuration within the outer tubular elongate member 130 and the inner tubular elongate member 160, such as illustrated in FIG. 2, to an extended configuration outside the inner tubular elongate member 160, such as illustrated in FIG. 3. In some aspects, the material layer 140 is formed from a flexible material which may crease or fold when in the stowed configuration. The material layer 140 may be non-compliant, semi-compliant, or compliant, such as determined by the particular use thereof. For instance, the material layer 140 may be formed of polyethylene terephthalate (PET). In some aspects, the material layer 140 of the tissue-separating tip element 150 of a tissue-separating end effector 110 formed in accordance with various principles of the present disclosure moves over the tissue-separating tip element 150 at the distal end 160d of the inner tubular elongate member 160 like an inverted balloon, such as a tunneling balloon. More particularly, the material layer 140 may have a periphery 142 (e.g., circumference, boundary, edge, etc.) which is coupled to the outer tubular elongate member 130 (e.g., along the exterior / outer surface of the distal end 130d of the outer tubular elongate member 130), and a radially-inward portion 144 which may be retractable within a lumen 131 of the outer tubular elongate member 130. The material layer 140 may be everted to extend distally out of the outer tubular elongate member 130. In some aspects, in contrast with a tunnelling balloon filled with fluid, the material layer 140 (e.g., the radially-inward portion 144) is engaged and pushed distally by the inner tubular elongate member 160 to be everted and extended out of the outer tubular elongate member 130, as may be appreciated with reference to FIGS. 2 and 3. In some aspects, the periphery 142 is fixedly coupled to the outer tubular elongate member 130 to not move with respect to the outer tubular elongate member 130 as the material layer 140 moves with respect to the inner tubular elongate member 160. For instance, the periphery 142 of the material layer 140 may be affixed to the distalmost end and/or the exterior of the outer tubular elongate member 130 in any of a variety of manners, such as adhesive, welding, mechanical affixing (such as with a crimping ring), etc., the present disclosure not being limited in this regard. In some aspects, the radially inward portion 144 of the material layer 140 extends within a lumen 161 of the inner tubular elongate member 160. The inner tubular elongate member 160 is retractable within the outer tubular elongate member 130 with the distalmost end 160e of the inner tubular elongate member 160 within the distal end 130d of the outer tubular elongate member 130 and optionally proximal to the distalmost end 130e of the outer tubular elongate member 130. If the tissue-separating tip element 150 is a separately formed element distal to the distalmost end 160e of the inner tubular elongate member 160, the tissue-separating tip element 150 may remain distal to the distalmost end 130e of the outer tubular elongate member 130 (such as if the tissue-separating tip element 150 forms a blunt tissue-contacting surface 152), or may be retracted within (e.g., proximal to at least the distalmost end 130e of) the outer tubular elongate member 130.

When the inner tubular elongate member 160 is in a retracted position, such as illustrated in FIG. 2, the inner tubular elongate member 160 may engage an inner surface of the material layer 140 closer to the periphery 142 than when the inner tubular elongate member 160 is distally extended out of the outer tubular elongate member 130, as may be appreciated upon comparison with the position illustrated in FIG. 3. As the inner tubular elongate member 160 is advanced from a retracted configuration (such as illustrated in FIG. 2), the material layer 140 is everted from within the inner tubular elongate member 160 to outside and along the exterior of the inner tubular elongate member 160. It will be appreciated that terms such as everted, unfurled, inverted, extended, etc., including various grammatical forms thereof, are usable interchangeably herein without intent to limit. The material layer 140 moves from the interior of the inner tubular elongate member 160 and over the tissue-separating tip element 150 (distalmost end 160e of the inner tubular elongate member 160 or the distal end of a separately formed tissue-separating tip element 150) to move along the contact surface 152 and along the circumferential exterior of the inner tubular elongate member 160.

As the material layer 140 moves from inside / within the inner tubular elongate member 160 to outside (and along the exterior of) the inner tubular elongate member 160, the material layer 140 moves over the tissue-separating tip element 150 (such as along the contact surface 152) and may be used to separate tissue in accordance with various principles of the present disclosure such as described herein. As may be appreciated, movement of the material layer 140 with respect to the inner tubular elongate member 160 upon distal movement of the inner tubular elongate member 160 provides the desired movement of material layer 140 along the tissue-separating tip element 150 and with respect to the contact surface 152 thereof to separate tissue contacted by at least the portion of the material layer 140 moving over the contact surface 152 of the tissue-separating tip element 150. As may be further appreciated, such movement from the interior to the exterior of the inner tubular elongate member 160 is analogous to the motion of a pulley, whereby the distance the material layer 140 moves (along the interior and the exterior of the inner tubular elongate member 160) is approximately twice the distance the inner tubular elongate member 160 is axially translated. As such, the movement of the material layer 140 with respect to the inner tubular elongate member 160 has a mechanical advantage which increases the speed of movement of the material layer 140 to achieve the desired tissue separation upon contact with tissue.

In some aspects, reciprocating axial motion of the inner tubular elongate member 160 with respect to the outer tubular elongate member 130 may be used to gently but effectively dissect tissue without a sharp cutting instrument. However, it will be appreciated that if the material layer 140 is not coupled to the inner tubular elongate member 160, such as in the example of an embodiment illustrated in FIGS. 2 and 3, proximal retraction of the inner tubular elongate member 160 may not cause the material layer 140 to return from outside the lumen 161 of the inner tubular elongate member 160 to inside the lumen 161 of the inner tubular elongate member 160 to effect a reciprocating motion. In accordance with various principles of the present disclosure, in some aspects, an elongate return element 170 is operably associated with the material layer 140 to at least facilitate return of the material layer 140 into the lumen 161 of the inner tubular elongate member 160. In some aspects, the elongate return element 170 is a pull wire, a tensioner, a string, a cord, a stylet, a filament, band, or any other elongate member configured and capable of being coupled at a distal end 170d thereof (see, e.g., FIGS. 2 and 3) to the material layer 140. Proximal movement of the elongate return element 170 upon proximal retraction of the inner tubular elongate member 160 moves the material layer 140 proximally and returns the material layer 140 into the lumen 161 of the inner tubular elongate member 160 as the inner tubular elongate member 160 is proximally retracted within the outer tubular elongate member 130. In some aspects, the elongate return element 170 moves proximally with the inner tubular elongate member 160 (e.g., upon proximal movement of the inner tubular elongate member 160) and does not need to be actively moved independently of and/or along with the inner tubular elongate member 160. In some aspects, tension is maintained on (e.g., constant tension is applied to) the elongate return element 170 so that the material layer 140 returns proximally into the inner tubular elongate member 160. In some aspects, tension is maintained on (e.g., constant tension is applied to) the elongate return element so that the material layer 140 maintains a relatively constant motion with respect to the distal end 160d of the inner tubular elongate member 160.

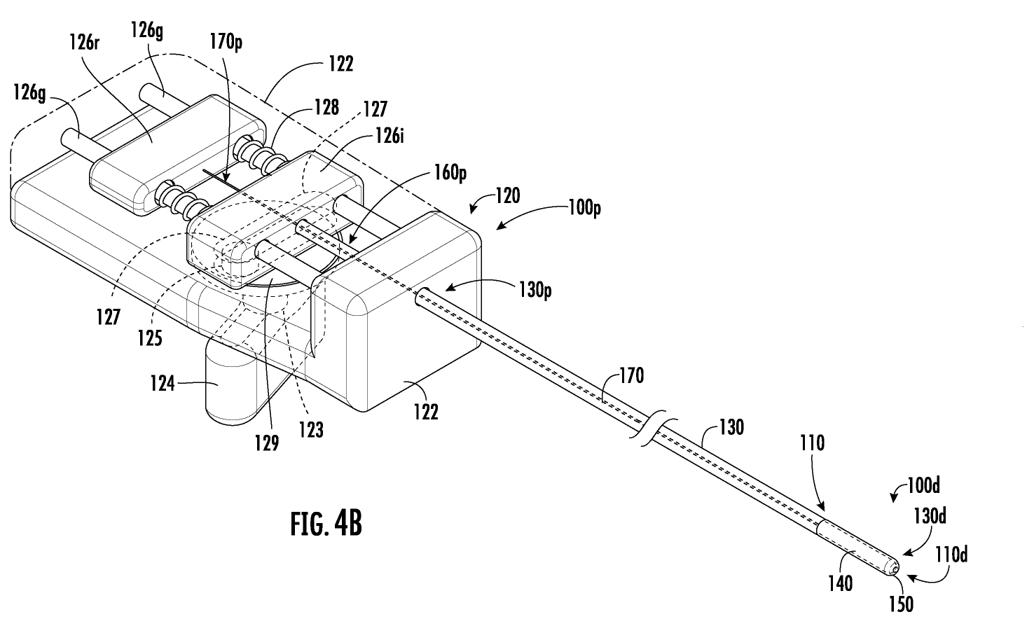

In accordance with various principles of the present disclosure, axial translation of the inner tubular elongate member 160 with respect to the outer tubular elongate member 130 may be controlled by a medical professional with the use of a control handle 120 with which the inner tubular elongate member 160 and the outer tubular elongate member 130 are operably associated. In some aspects, such as noted above, the outer tubular elongate member 130 extends proximally from the tissue-separating tip element 150 of the tissue separating system 100 to the control handle 120 as illustrated in FIG. 1. Additionally or alternatively, in some aspects, the inner tubular elongate member 160 extends proximally from the tissue-separating tip element 150 of the tissue separating system 100 to the control handle 120, as illustrated in FIGS. 4A and 4B, to control movement of the material layer 140. In the example of an embodiment of a control handle 120 illustrated in FIGS. 1, 4A, and 4B, the proximal end 130p of the outer tubular elongate member 130 is coupled to the control handle 120. In some aspects, the outer tubular elongate member 130 is coupled to the housing 122 of the control handle 120 to move with the housing 122, and is not movable with respect to the housing 122. In contrast, the inner tubular elongate member 160 is operably associated with an actuator 124 accessible by a medical professional to effect movement of the inner tubular elongate member 160, such as with respect to the housing 122 of the control handle 120. The actuator 124 may be manually accessible by a medical professional (e.g., outside the housing 122 and graspable and manipulable, such as rotated or slid, by the medical professional) or may even be a switch to control an automated mechanism.

In some aspects, to control movement of the inner tubular elongate member 160 with respect to the outer tubular elongate member 130, the proximal end 160p of the inner tubular elongate member 160 extends proximal to the proximal end 130p of the outer tubular elongate member 130. As illustrated in FIGS. 4A and 4B, the inner tubular elongate member 160 is operably associated with a first actuator block 126i of the control handle 120. The first actuator block 126i of the illustrated example of an embodiment is longitudinally translatable to advance or retract the inner tubular elongate member 160. More particularly, in the example of an embodiment illustrated in FIGS. 4A and 4B (showing the housing 122 in phantom), the first actuator block 126i is operably associated with the actuator 124, and is movable (e.g., axially translatable) along one or more actuator block guides 126g by movement of the actuator 124. In some aspects, the actuator 124 is rotatable to longitudinally translate the first actuator block 126i to move axially and to longitudinally translate / axially move the inner tubular elongate member 160. Such actuation may be achieved in a variety of manners. In the example of an embodiment illustrated in FIGS. 4A and 4B, a projection on one of the actuator 124 or the first actuator block 126i rides along a cam surface on the other of the actuator 124 or the first actuator block 126i so that rotation of the actuator 124 causes longitudinal translation of the first actuator block 126i and thus the inner tubular elongate member 160. For instance, in the illustrated example of an embodiment, a projection 125 extends from the actuator 124 into a cam surface 127 defined in the first actuator block 126i. As the actuator 124 is rotated, the projection 125 is moved by the cam surface 127 to move the inner tubular elongate member 160 between a retracted position, as illustrated in FIG. 4A, and an extended position, such as illustrated in FIG. 4B. Such configuration allows for a generally continuous motion to effect reciprocation (back and forth movement between retracted and extended positions) of the tissue-separating tip element 150. For instance, the actuator knob 124 may be rotated continuously either clockwise or counterclockwise (i.e., in the same selected direction) to achieve a reciprocating movement of the tissue-separating tip element 150. In some aspects, the projection 125 is mounted radially away from a center of a generally circular (or oval) base 129, and the base 129 is coupled to the actuator 124 via a shaft 123 of the actuator 124. Rotation of the actuator 124 and the actuator shaft 123 thus rotate the base 129 to move the projection 125 in a circular, elliptical, or other configuration (typically nonlinear) with respect to a generally linear / straight cam surface 127 to axially move the inner tubular elongate member 160 (operably coupled with the first actuator block 126i and thus with the cam surface 127). It will be appreciated that the first actuator block 126i may be longitudinally translated in any of a variety of alternative manners, such as to achieve a reciprocating movement. For instance, sliders, motorized controllers, or other actuators known to those of ordinary skill in the art may be used without detracting from the principles of the present disclosure.

In accordance with various principles of the present disclosure, the elongate return element 170 may also extend proximally to be operably associated with the control handle 120. such as to be controlled via the control handle 120. In some aspects, the proximal end 170p of the elongate return element 170 is operably associated with the actuator 126 so that actuation of the actuator 126 effects the desired movement of the elongate return element 170, such as to retract the material layer 140. For instance, in the example of an embodiment illustrated in FIGS. 4A and 4B, the proximal end 170p of the elongate return element 170 extends proximal to the proximal end 160p of the inner tubular elongate member 160 to be operably associated with a second actuator block 126r of the control handle 120. In some aspects, the second actuator block 126r is movably mounted along the one or more actuator block guides 126g. The second actuator block 126r of the illustrated example of an embodiment is longitudinally translatable to advance or retract the elongate return element 170. For instance, in the illustrated example of an embodiment, the second actuator block 126r is operably associated with the first actuator block 126i to move as the first actuator block 126i is actuated by the actuator 126. In accordance with various principles of the present disclosure, if the elongate return element 170 maintains constant tension on the material layer 140, a biasing element 128 (e.g., a coil spring) may be used to bias the second actuator block 126r away from the first actuator block 126i to maintain tension on the elongate return element 170.

In use, the tissue separating system 100 may be deployed through a working channel of a medical scope, such as with the inner tubular elongate member 160 retracted within the outer tubular elongate member 130. The tissue-separating tip element 150 may be advanced to an incision at a treatment / target site within the patient, such as an incision made prior to delivery of the tissue-separating end effector 110 to give access to the tissue to be separated (e.g., a submucosal tissue layer) at the target site. The inner tubular elongate member 160 may then be pushed distally out of the outer tubular elongate member 130, moving the material layer 140 with respect to the inner tubular elongate member 160 and with respect to the tissue-separating tip element 150. As noted above, the material layer 140 may move similar to how a pully moves. For instance, for every 1 mm of distal movement of the inner tubular elongate member 160, 2 mm of the material layer 140 is deployed. As may be appreciated, the elongate return element 170 moves at a rate of approximately 2:1 with respect to the movement of the inner tubular elongate member 160. The elongate return element 170 may be passively pulled distally as the inner tubular elongate member 160 is advanced distally. In some aspects, the material layer 140 reciprocates in opposite directions over the tissue-separating tip element 150, such as by advancing and retracting the inner tubular elongate member 160, such as described above. Such distal and proximal movements of the inner tubular elongate member 160, material layer 140, and elongate return element 170 can be performed in quick short succession in an oscillatory fashion or with single larger movements. Once the desired deployment depth is reached, the inner tubular elongate member 160 and the elongate return element 170 may be moved proximally to return the material layer 140 into the inner tubular elongate member 160 (a retracted configuration) and the inner tubular elongate member 160 back to a stowed position within the outer tubular elongate member 130.

In some aspects, the elongate return element 170 has a lumen defined therethrough, with the material layer 140 secured to the exterior of the elongate return element 170 (e.g., around and to the circumference of the distal end 170d of the elongate return element 170). Such securing may be achieved in any of a variety of manners such as known to those of ordinary skill in the art, such as described above with reference to securing the material layer 140 to the exterior of the outer tubular elongate member 130. The lumen of a tubular elongate return element 170 may allow passage of an instrument distal to the material layer 140. For instance, a sharper cutting instrument 180 (e.g., one or more of a hot knife, an electrocautery knife, or an injection needle) may be advanced through the elongate return element 170 to extend the cutting edge 182 thereof distal to the tissue-separating end effector 110. The cutting edge 182 of the cutting instrument 180 may be used to assist in creating an initial incision through which the tissue-separating end effector 110 is advanced. Additionally or alternatively, the cutting edge 182 of the cutting instrument 180 may be used to cut remaining fibers, vessels, etc., after tissue separation by the tissue-separating end effector 110 of the present disclosure is completed (e.g., the final dissection of an endoscopic submucosal dissection procedure). In some aspects, other materials (e.g., fluid) may be transported through the lumen of the elongate return element 170.

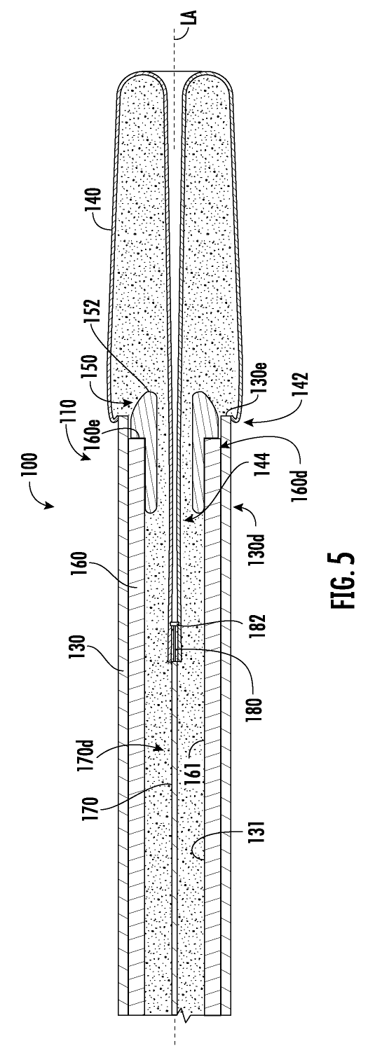

In some aspects, a tissue-separating end effector 110 formed in accordance with various principles of the present disclosure may also be used to separate tissue by expansion of the tissue-separating end effector 110. For instance, in some aspects, the inner tubular elongate member 160 may be fluidly coupled with a fluid supply (e.g., a syringe or other fluid supply known to those of ordinary skill in the art, the present disclosure not being limited in this regard) and filled with fluid (e.g., saline, air, etc.) to fill the material layer 140, such as illustrated in FIG. 5. The inner tubular elongate member 160 may be proximally withdrawn without withdrawing the elongate return element 170, to allow the material layer 140 to expand (e.g., to expand the cross-sectional area thereof) to separate tissue. For instance, the first actuator block 126i may be disengaged from the second actuator block 126r, such as by using a system of gears, springs, pulleys, etc., as known to those of ordinary skill in the art, to allow proximal withdrawal of the inner tubular elongate member 160 without proximally withdrawing the elongate return element 170. In some aspects, the material layer 140 may be inflated like a ballon to make larger channels in the tissue being separated than may be achieved by the cross-sectional dimension of the tissue-separating tip element 150 and/or the surface area of the contact surface 152 prior to expansion of the material layer 140. As may be appreciated, if the material layer 140 is to be expanded in such manner, the material layer 140 may be affixed to the tubular elongate member 130 in a fluid tight manner such as known to those of ordinary skill in the art.

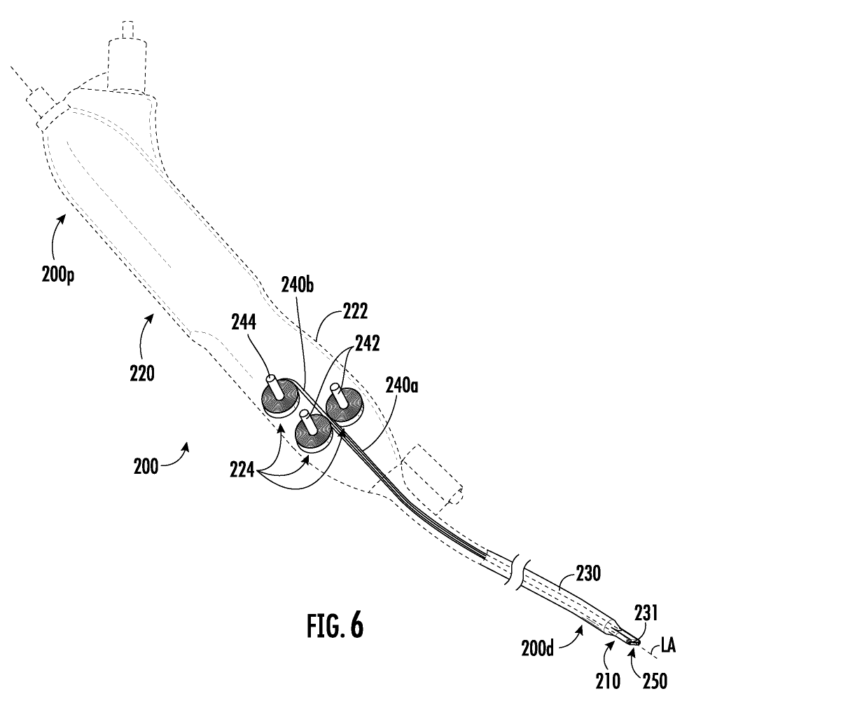

Various principles of the present disclosure with regard to moving a material with respect to tissue to dissect the tissue may be applied in various alternative configurations. For instance, the example of an embodiment of a tissue separating system 200 illustrated in FIG. 6 has, at a distal tissue-separating end 200d thereof, a tissue-separating end effector 210 with a tissue-separating tip element 250 over which an elongated material layer 240 moves. A control handle 220 at the proximal control end 200p controls movement of the elongated material layer 240 with respect to (e.g., over the distal-facing surface of) the tissue-separating tip element 250. Contact of the moving elongated material layer 240 with tissue causes separation, such as dissection, of the tissue, such as in a manner as described above.

In some aspects, the example of an embodiment of a tissue separator 200 illustrated in FIG. 6 utilizes an elongated material layer 240 extending along, such as within, a tubular elongate member 230 extending longitudinally along a longitudinal axis LA of the tissue separating system 200 (e.g., distally from the control handle 220 to the tissue-separating end effector 210). The elongated material layer 240 may be in the form of a band, strip, belt, etc., having a dimension along the longitudinal axis LA of the tissue-separating system 200 longer than a dimension transverse to (not parallel to) the longitudinal axis LA. In some aspects, the elongated material layer 240 extends longitudinally along the longitudinal axis LA of the tissue separating system 200 between the tissue-separating end effector 210 and the control handle 220 of the tissue separating system 200. In some aspects, the tissue-separating tip element 250 is formed along a distal end 230d of the tubular elongate member 230. In some aspects, the elongated material layer 240 has a width substantially equal to or less than a diameter (outer or inner) of the tubular elongate member 230.

In some aspects, an additional elongate element movable within and with respect to the tubular elongate member 230 (such as the inner tubular elongate member 160, movable within the outer tubular elongate member 130 of the tissue separating system 100 illustrated in FIG. 1) is not used to move the elongated material layer 240 with respect to the tissue-separating tip element 250 of the tissue-separating end effector 210. In some aspects, an actuator 224 (schematically illustrated in FIG. 6) is operably associated with the housing 222 of the control handle 220 and with the elongated material layer 240 to move the elongated material layer 240 with respect to the tissue-separating tip element 250 of the tissue-separating end effector 210. In some aspects, the actuator 224 is substantially directly coupled with the elongated material layer 240 to effect movement of the elongated material layer 240. As used herein, “directly” coupled is intended to include coupling elements used to achieve the desired coupling, but not structural elements of the tissue-separating end effector 210 which may have functions in addition to coupling. In some aspects, the elongated material layer 240 extends from the tissue-separating end effector 210 proximally to the control handle 220 at the proximal end 200p of the tissue separator 200 to be operably coupled to the actuator 224. such as within the control handle 220. In some aspects, the elongated material layer 240 extends from a first end 240a (e.g., a free end) to a second end 240b (e.g., a free end). In some aspects, each end 240a, 240b of the elongated material layer 240 is coupled to a different actuator component and/or element about which the elongated material layer 240 may be stowed, gathered, wound, stored, spooled, etc. (such terms being usable interchangeably herein without intent to limit), as discussed in further detail below. Alternatively, the elongated material layer 240 may be formed in a continuous loop. In some aspects, the elongated material layer 240 is not coupled to the tubular elongate member 230 (as in the tissue separating system 100 illustrated in FIG. 1), but, instead, moves with respect to the tubular elongate member 230, such as substantially continuously, as described in further detail below.

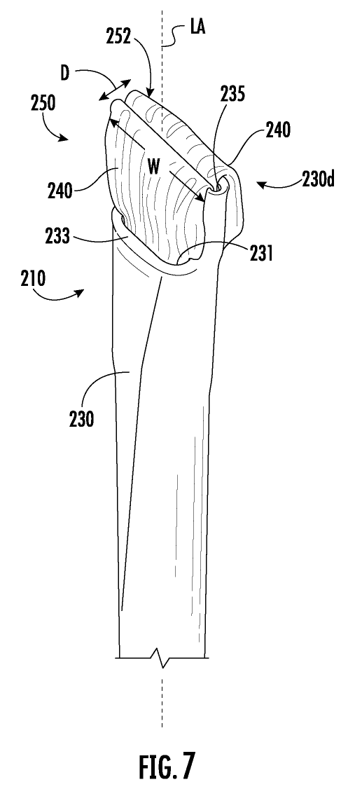

Further details of an example of an embodiment of tissue-separating tip element 250 of a tissue separator 200 as illustrated in FIG. 6 are illustrated in FIG. 7. As may be appreciated with reference to FIG. 7, the elongated material layer 240 extends from within a lumen 231 defined within the tubular elongate member 230, out of the lumen 231, over a distal-facing surface 252 of the tissue-separating tip element 250, and back into the lumen 231. In some aspects, the tissue-separating tip element 250 is fixedly coupled with the tubular elongate member 230 and does not move relative to the tubular elongate member 230 to move the elongated material layer 240.

In some aspects, the tissue-separating tip element 250 is formed by a distal end 230d of the tubular elongate member 230. For example, in the example of an embodiment illustrated in FIG. 7, the tissue-separating tip element 250 may be formed by crushing or crimping the distal end 230d of the tubular elongate member 230 laterally inwardly (toward the longitudinal axis LA). Additionally, axially-extending cut-outs 233 may be formed radially through the crimped portion of the wall defining the tubular elongate member 230. In the illustrated example of an embodiment, the elongated material layer 240 extends distally out of the lumen 231 of the tubular elongate member 230 through the slit opening 235 defined between opposed crimped sides of the distal end 230d of the tubular elongate member 230 which form the tissue-separating tip element 250, and over the contact surface 252 of the tissue-separating end effector 210. The elongated material layer 240 then returns into the lumen 231 via the cut-outs 233. The elongated material layer 240 may include two layers of material which separate as they exit the slit opening 235, and move in opposite directions to return proximally into the lumen 231 through oppositely positioned cut-outs 233, as described in further detail below.

In some aspects, the crimped configuration of the tissue-separating end effector 210 allows for long contact lines of the elongated material layer 240 with tissue along the contact surface 252 defined by the tissue-separating end effector 210, such as in comparison with the contact surface 152 of the tissue-separating end effector 110 described above. For instance, in some aspects, the configuration of the tissue-separating end effector 210 allows for contact of flatter areas of the elongated material layer 240 with tissue than would be achieved if the elongated material layer 240 were extended over a curved surface (such as the curved exterior of the tubular elongate member 230 proximal to the crimped distal end 230d which forms the tissue-separating end effector 210). In some aspects, the configuration of the elongated material layer 240 as it extends over the contact surface 252 may be somewhat planar, akin to a shovel, which may dig or otherwise extend into tissue to be separated. It will be appreciated that a flatter, more planar configuration of the elongated material layer 240, as in the example of an embodiment illustrated in FIGS. 6 and 7, may be desirable for ESD procedures, whereas a more curved material layer 140, as in the example of an embodiment illustrated in FIGS. 1 - 5, may be desirable for POEM procedures. Moreover, it will be appreciated that unlike the material layer 140 of the above-described tissue-separating system 100 which may crease or fold within the tissue-separating system 100 (e.g., within the outer tubular elongate member 130), the elongated material layer 240 may extend within and through the tissue-separating system 200 (e.g., through the tubular elongate member 230) without (or at least with minimal) folding, creasing, or otherwise. In particular, if the width of the elongated material layer 240 is less than the inner diameter of the tubular elongate member 230, the elongated material layer 240 may remain substantially planar through as well as outside the tubular elongate member 230, such as along the tissue-separating end effector 210. As such, the outer, tissue-contacting surface of the elongated material layer 240 may be textured (e.g., roughened; provided with embossed or recessed areas, such as patterns; or otherwise not flat or planar) to increase local tissue displacement and/or to increase friction with respect to tissue to be separated by the movement of the elongated material layer 240. In some aspects, the relatively constant surface area of the elongated material layer 240 as the elongated material layer 240 moves along the tissue-separating system 200 allows for maximized contact area of the elongated material layer 240 with tissue.

In some aspects, the contact surface 252 formed by crimping the distal end 230d of the tubular elongate member 230 defines an elongated contact surface 252 having a dimension along the width W of the elongated material layer 240 (generally transverse to the direction of movement of the elongated material layer 240) greater than the distance D between which the distally-extending portion of the elongated material layer 240 is separated from the proximally-extending portion of the elongated material layer 240 as the elongated material layer 240 moves over the contact surface 252. As may be appreciated, the elongated shape of the contact surface 252 of the tissue-separating tip element 250 allows the tissue-separating tip element 250 to separate tissue in a chosen plane or axis along the width W of the elongated material layer 240 and the contact surface 252 (generally at an angle, not 0° or 180°, with respect to the longitudinal axis LA of the tissue-separating tip element 250), depending on the rotation of the tissue-separating tip element 250 with respect to tissue (such as about the longitudinal axis LA of the tissue-separating tip element 250).

As may be appreciated with reference to the example of an embodiment illustrated in FIG. 6¸ in some aspects, the elongated material layer 240 may be in a stowed configuration, such as rolled about axles 242, 244 (e.g., like a roll of tape), proximal to the tissue-separating end effector 210. For instance, the first end 240a of the elongated material layer 240 may be stowed within the housing 222 of the control handle 220, such as by being wound about a return or take-up axle 242. Additionally or alternatively, the second end 240b of the elongated material layer 240 may be stowed within the housing 222 of the control handle 220, such as by being wound about a feed or supply axle 244. As the actuator 224 distally advances the elongated material layer 240 to the tissue-separating end effector 210, the elongated material layer 240 is unrolled from its stowed configuration (e.g., spooled about the supply axle 244), moves over the tissue-separating tip element 250, and is returned proximally to the take-up axle 242, such as to be spooled about the take-up axle 242. The elongated material layer 240 may be arranged in a rotary system, such as illustrated in FIG. 6, or in a continuous, single loop. In some aspects, the elongated material layer 240 moves in one direction. In some aspects, the actuator 224 may be a knob or other user-engageable element operably coupled with the take-up axle 242 to proximally pull on the elongated material layer 240. Such proximal movement of the elongated material layer 240 towards the first end 240a causes the elongated material layer 240 to unwind from the supply axle 244 and move distally over the tissue-separating tip element 250 to return proximally to the take-up axle 242. The elongated material layer 240 may be wound or stowed about the take-up axle 242. Optionally, the elongated material layer 240 may be moved in an opposite direction, such as to achieve a reciprocating motion of the elongated material layer 240 with respect to tissue . For instance, the actuator 224 may be operably coupled with the supply axle 244 to cause the supply axle 244 to proximally pull the elongated material layer 240 toward the supply axle 244 and distally pull the elongated material layer 240 from the take-up axle 242, achieving movement with respect to the tissue-separating tip element 250 opposite the movement resulting from movement of the elongated material layer 240 toward the take-up axle 242.

In some aspects, such as illustrated in FIGS. 6 and 7, two elongated material layers 240 move across the distally-facing contact surfaces 252 of the tissue-separating tip element 250 of the tissue-separating end effector 210 in opposite directions, causing tissue separation (e.g., causing tissue fiber to elongate and then break). In some aspects, the movements of the two elongated material layers 240 are generally mirror images, with the elongated material layers 240 moving in opposite directions with respect to the tissue-separating tip element 250 of the tissue-separating end effector 210. For the sake of convenience, and without intent to limit, the same reference characters are used for the two elongated material layers 240. Moreover, for the sake of brevity, and without intent to limit, the above descriptions of the configuration and movements of one of the elongated material layers 240 as described above is applicable, mutatis mutandis, to the other of the elongated material layers 240. However, it will be appreciated that in the illustrated example of an embodiment, both elongated material layers 240 are fed from a common supply axle 244 (e.g., are wound together about a common spool), and fed together distally to the tissue-separating end effector 210, where the elongated material layers 240 are separated to move in opposite directions and then proximally to separate take-up axles 242. More particularly, in some aspects, the elongated material layers 240 move distally together through the lumen 231 of the tubular elongate member 230 and out of the slit opening 235. The elongated material layers 240 then move in opposite directions into cut-outs 233 on opposite sides of the tubular elongate member 230¸ and return (typically alongside each other, although optionally spaced apart) proximally the lumen 231 of the tubular elongate member 230 to respective take-up axles 242. It will be appreciated that the separation of the two elongated material layers 240 from the slit opening 235 at the contact surface 252 of the tissue-separating end effector 210 may define a quick dislocation line at which tissue contacted by the contact surface 252 is separated by the tissue-separating end effector 210. Use of a tissue-separating system 200 such as illustrated in FIGS. 6 and 7 may be similar to the use of a tissue-separating system 100 such as illustrated in FIGS. 1, 2, 3, 4A, 4B, and 5, reference being made to the above description of the tissue-separating system 100 as applying mutatis mutandis to the tissue-separating system 200.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. It is to be understood by one of ordinary skill in the art that the present discussion is a description of illustrative examples of embodiments only, and is not intended as limiting the broader aspects of the present disclosure. Various further benefits of the various aspects, features, components, and structures of tissue separating devices, systems, and methods such as described above, in addition to those discussed above, may be appreciated by those of ordinary skill in the art. It therefore will be appreciated that the foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and / or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A tissue separating device having a tissue-separating end effector extending along a longitudinal axis, said tissue separating device comprising:
an outer tubular elongate member having a distal end along the tissue-separating end effector of said tissue separating device and a proximal end;
an inner tubular elongate member axially translatable within the outer tubular elongate member and having a distal end and a proximal end;
a layer of material having a periphery coupled to said outer tubular elongate member and a radially-inward portion radially inward of the periphery and extending over the distal end of said inner tubular elongate member; and
a tissue-separating tip element defining a contact surface extending transverse to the longitudinal axis;
wherein:
movement of said inner tubular elongate member with respect to said outer tubular elongate member moves said layer of material with respect to said outer tubular elongate member and said tissue-separating tip element; and
movement of said layer of material with respect to the contact surface of said tissue-separating tip element causes separation of tissue contacted by said layer of material along the contact surface.

2. The device of claim 1, wherein the periphery of said layer of material is fixedly coupled with respect to said outer tubular elongate member.

3. The device of claim 1, further comprising a control handle operably associated with the proximal end of said outer tubular elongate member and the proximal end of said inner tubular member.

4. The device of claim 3, wherein said control handle comprises an actuator operably coupled to said inner tubular elongate member to axially translate said inner tubular member with respect to said outer tubular elongate member to cause said layer of material to move with respect to said inner tubular elongate member.

5. The device of claim 1, further comprising an elongate return element coupled to the radially-inward portion of said layer of material and configured to pull said layer of material proximally as said inner tubular elongate member is moved proximally within said outer tubular elongate member.

6. The device of claim 5, wherein said elongate return element defines a lumen therethrough and extending through said layer of material.

7. The device of claim 1, wherein said tissue-separating end effector further comprises a tissue-separating tip element.

8. The device of claim 7, wherein said tissue-separating tip element defines a blunt end defining the contact surface of said tissue-separating end effector.

9. The device of claim 7, wherein said tissue-separating tip element is provided along a distalmost end of said outer tubular elongate member.

10. The device of claim 9, wherein said tissue-separating tip element is separate from the distalmost end of said outer tubular elongate member.

11. A tissue separating device having a tissue-separating end effector extending along a longitudinal axis, said tissue separating device comprising:
an outer tubular elongate member having a distal end along the tissue-separating end effector of said tissue separating device and a proximal end;
an inner tubular elongate member axially translatable within the outer tubular elongate member and having a distal end and a proximal end;
a layer of material extending over the distal ends of said outer tubular elongate member and said inner tubular elongate member;
a tissue-separating tip element defining a contact surface extending transverse to the longitudinal axis; and
an elongate return element having a distal end coupled to said layer of material and a proximal end;
wherein:
distal movement of said inner tubular member moves said layer of material distally and with respect to said tissue-separating tip element;
said inner tubular member is movable proximally without moving said layer of material proximally; and
proximal movement of said elongate return element moves said layer of material distally and with respect to the contact surface of said tissue-separating tip element to cause separation of tissue contacted by said layer of material along the contact surface.

12. The device of claim 11, wherein said layer of material is fixedly attached with respect to said inner tubular member and movable with respect to said outer tubular member.

13. The device of claim 11, further comprising a control handle operably associated with the proximal end of said outer tubular elongate member, the proximal end of said inner tubular member, and the proximal end of said elongate return element.

14. The device of claim 13, wherein said control handle further comprises a first actuator block operably associated with the proximal end of said inner tubular member, and a second actuator block operably associated with the proximal end of said elongate return element and movable with respect to said first actuator block.

15. The device of claim 14, wherein movement of said layer of material with respect to the contact surface of said tissue-separating tip element and movement of said second actuator block is faster than movement of said first actuator block.

16. A method of separating tissue, said method comprising:
providing a tissue separator having a tissue-separating end effector with a tissue-separating tip element and a layer of material having a first portion fixedly coupled to a first element of the tissue-separator and a second portion fixedly coupled to a second element of the tissue-separator; and
moving the first element relative to the second element to move the layer of material with respect to the tissue-separating tip element.

17. The method of claim 16, further comprising axially moving the second element with respect to the first element to move the layer of material with respect to the tissue-separating tip element.

18. The method of claim 17, wherein the first element is an outer tubular elongate member, the tissue separator further having an inner tubular elongate member movable with respect to the outer tubular elongate member, said method further comprising moving the inner tubular elongate member distally with respect to the outer tubular elongate member to move the layer of material from within a lumen of the inner tubular elongate member to outside the inner tubular elongate member, and moving the second element proximally with respect to the outer tubular elongate member to retract the layer of material into the inner tubular elongate member.

19. The method of claim 17, further comprising moving the layer of material distally from a proximal end of the tissue separator to a distal end of the tissue separator, over a tissue contact surface of the tissue-separating tip element, and proximally to the proximal end of the tissue separator.

20. The method of claim 19, wherein the first portion of the layer of material is fixedly coupled to the first element and the second portion of the layer of material is fixedly coupled to the second element, said method further comprising moving the first element proximally with respect to the second element to move the layer of material over and with respect to the tissue-separating tip element.
